(19) United States
(12) Patent Application Publication
(10) Pub. No.: US 2013/0303408 A1
(43) Pub. Date: Nov. 14, 2013

(54) ASSEMBLIES FOR MULTIPLEX BINDING ASSAYS

(71) Applicant: Pierre F. Indermuhle, Berkeley, CA (US)

(72) Inventor: Pierre F. Indermuhle, Berkeley, CA (US)

(21) Appl. No.: 13/851,228

(22) Filed: Mar. 27, 2013

Related U.S. Application Data
(63) Continuation of application No. 13/040,433, filed on Mar. 4, 2011, now abandoned.
(60) Provisional application No. 61/310,824, filed on Mar. 5, 2010.

(51) Int. Cl. G01N 33/543; C12Q 1/68

(52) U.S. Cl. CPC G01N 33/54366; C12Q 1/6837
USPC 506/37

(57) ABSTRACT

Assay plate assemblies are disclosed. The assemblies include an assay plate that has a top side, a bottom side, and at least one well accessible from the top side of the plate. The well includes a side surface and a bottom surface, with at least one secondary container protruding through the bottom surface and into an interior volume of the well. The assemblies further include a dispenser plate that is adapted to be positioned adjacent to the top side of the assay plate. The dispenser is further configured to provide one or more reagents to one or more secondary containers of at least one well of the assay plate.

### CROSS-REFERENCE TO RELATED APPLICATIONS

This application is a continuation of U.S. patent application Ser. No. 13/040,433, filed Mar. 4, 2011, which claims priority to, and incorporates by reference, U.S. provisional application Ser. No. 61/310,824, which was filed on Mar. 5, 2010.

### FIELD OF THE INVENTION

The field of the present invention relates to assemblies for use in multiplex binding assays. More particularly, the field of the present invention relates to dispensers and assay plates, which may comprise a plurality of primary wells (with one or more secondary containers included within each of such wells), which may be used to carryout multiplex binding assays.

### BACKGROUND OF THE INVENTION

A multiplex assay is a type of procedure that simultaneously measures (or otherwise detects and/or analyzes in some fashion) multiple analytes—in a single assay. Multiplex assays have been used in order to detect or quantify various biomolecules in a particular sample, such as mRNAs, proteins, antibodies, and other biomolecules. Multiplex assay formats are often beneficial, insofar as such formats can provide a significant reduction in assay costs, on a cost-per-analyte basis. In addition, such formats significantly increase the amount (and often types) of information that can be extracted from each sample, particularly on a per-sample-volume basis.

Despite the significant utility of multiplex assay formats, present platforms do not allow for the dispensing of a specific secondary binding agent (i.e., the detection agent) to each immobilized target, in order to reduce cross-reactivity (which leads to false positive results). This drastically limits the types of assays that may be combined in a multiplex fashion (and, more particularly, the combination of analytes that may be measured or detected in a single assay format). In addition, current platforms do not allow individual assay conditions, e.g., sample dilutions, buffer types, incubation times, etc., to be optimized. Accordingly, a continuing need exists for new and improved multiplex binding assay assemblies and methods of use thereof.

### SUMMARY OF THE INVENTION

According to certain aspects of the present invention, assay plate assemblies are provided. The assemblies include an assay plate that has a top side, a bottom side, and at least one well accessible from the top side of the plate. The at least one well includes a side surface and a bottom surface, with at least one secondary container (and, optionally, multiple secondary containers) protruding through the bottom surface and into an interior volume of the well. The assemblies further include a dispenser plate that is adapted to be positioned adjacent to the top side of the assay plate. The dispenser is further configured to provide one or more reagents, vis-a-vis one or more reservoirs included within the dispenser, to one or more secondary containers of at least one well of the assay plate.

According to certain related aspects of the present invention, assay plate assemblies are provided, which include an assay plate that has a top side, a bottom side, and a plurality of wells accessible from the top side of the plate, such as 96, 384, or 1536 wells. Similar to the embodiment described above, the wells include a side surface and a bottom surface, with at least one secondary container, and preferably a plurality of secondary containers, protruding through the bottom surface and into an interior volume of each well. Similar to the embodiment described above, the assemblies further include a dispenser plate that is adapted to be positioned adjacent to the top side of the assay plate, which is configured to provide one or more reagents to the secondary containers included within certain wells of the assay plate. According to certain embodiments, similarly configured assay plates may be stacked upon each other, with the secondary containers included within the wells of such assay plates being in fluid communication with each other, which creates a type of interconnected capillary system between the plates (created by the secondary containers of a first plate being stacked on top of the secondary containers of a second plate).

According to additional aspects of the present invention, the assay plates described herein, with or without the dispensers described below, are encompassed by the present invention.

According to yet further aspects of the present invention, methods of using the assay plate assemblies described herein, for carrying out multiplex binding assays, are encompassed by the present invention.

The above-mentioned and additional features of the present invention are further illustrated in the Detailed Description contained herein.

### BRIEF DESCRIPTION OF THE FIGURES

FIG. 1 is a partial top side view of an assay plate that comprises a plurality of secondary containers located in the bottom of a plurality of individual wells. The secondary containers exhibit protruding edges (at the top ends of such secondary containers), which allow these secondary containers to be provided with a reagent by introducing such edges into the reservoirs of a dispenser, as described further below.

FIG. 2 is a partial bottom view of the assay plate of FIG. 1, showing multiple wells that include a plurality of secondary containers (with protruding edges at the bottom ends of such secondary containers) that transverse the bottom surface of each well.

FIG. 3 is a partial top side view of a 96-well plate, which includes a plurality of secondary containers in each well, which further shows a dispenser located on top of (above) a single column of wells in the assay plate, which may be used to provide the secondary containers within such wells with a desired reagent (or set of reagents).

FIG. 4 is a cross-sectional diagram showing a well with two secondary containers located therein, and a dispenser aligned with and placed on top of the plate for dispensing a separate reagent into each of the two secondary containers.

FIG. 5 is a top side perspective view of three wells of a capillary-in-a-well dispensing bar.

FIG. 6 is a bottom, side view of a capillary-in-a-well plate, with the secondary containers being entirely filled with a reagent (up to the bottom edges thereof), with the reagent being retained therein via capillary forces.

FIG. 7 is a side view of two capillary-in-a-well plates (or bars) stacked on top of each other. As illustrated therein, a liquid reagent is allowed to fill the secondary containers without leaking at the intersection between the two plates (i.e., between the secondary containers of each plate). The secondary containers are filled with a volume of liquid that is entirely containd therein (up to the bottom edges of the secondary containers) via capillary forces.

### DETAILED DESCRIPTION OF THE INVENTION

The following will describe, in detail, several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used and made without departing from the scope and spirit of the invention.

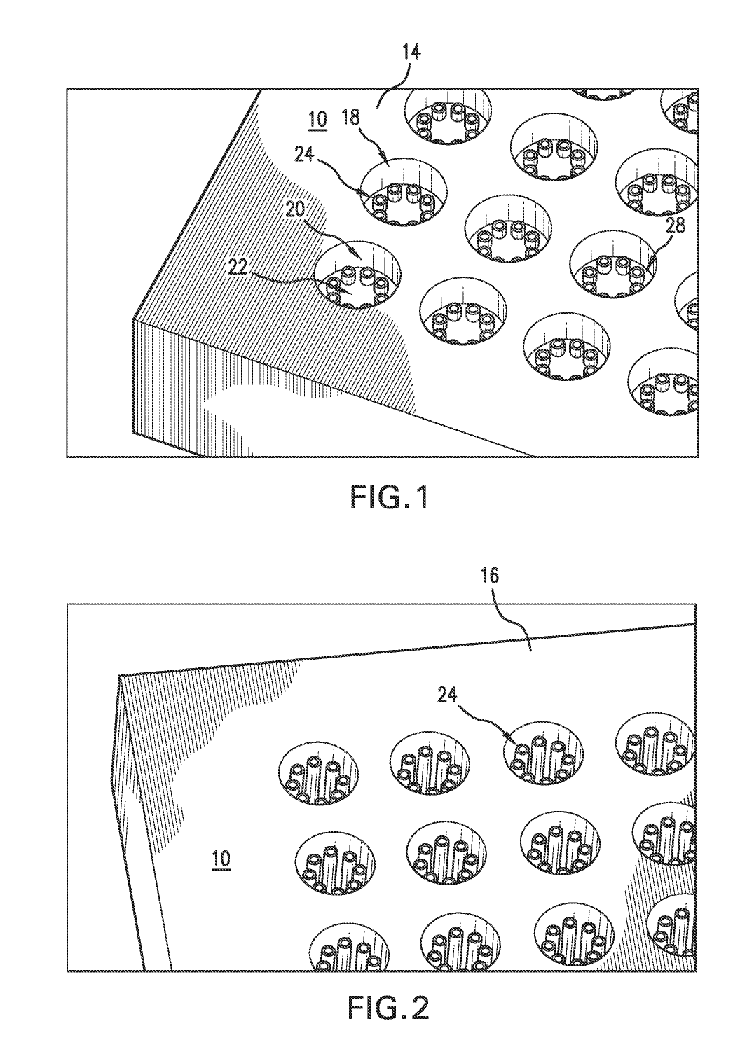

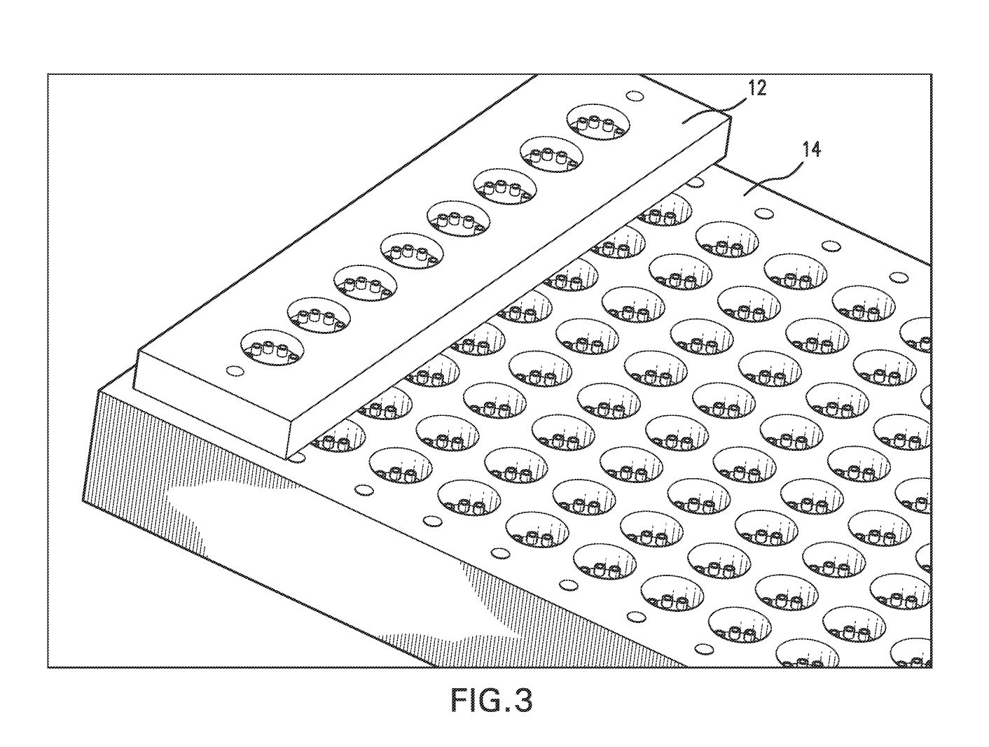

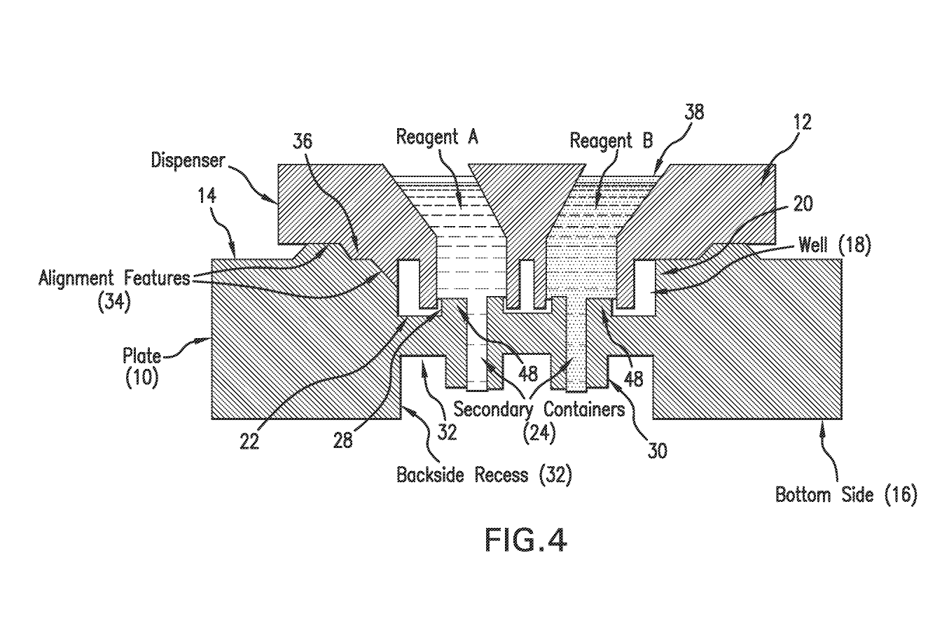

Referring now to FIGS. 1-7, the invention provides assay plate assemblies that may be used to carry out multiplex binding assays. The assemblies generally include an assay plate 10 and a dispenser 12. The assay plate 10 has a top side 14, a bottom side 16, and at least one well 18 accessible from the top side 14 of the plate. The well includes a side surface 20 and a bottom surface 22, with at least one secondary container 24 protruding through the bottom surface 22 and into an interior volume of the well 18. The assemblies further include a dispenser plate 12 that is adapted to be positioned adjacent to the top side 14 of the assay plate 10, as illustrated in FIGS. 3 and 4. The dispenser 12 is further configured to provide one or more reagents (FIG. 4) to one or more secondary containers 24 of at least one well 18 of the assay plate 10.

According to certain preferred embodiments, the secondary containers 24 consist of a capillary tube 26 (FIG. 7), which may span a distance that is equal to the thickness (or approximately equal to the thickness) of the assay plate 10, with the “thickness” of the assay plate 10 being the distance between the top side 14 and bottom side 16 thereof. According to certain embodiments of the invention, the secondary containers 24 will begin (at the top ends thereof) at a location 28 that protrudes through the bottom surface 22 and into an interior volume of each well 18, and end at a location 30.

In certain embodiments, the invention provides that the secondary containers 24 may extend beyond a bottom side 16 of the assay plate 10 (FIGS. 6 and 7). According to such embodiments, the distance between the top end 28 of each secondary container 24, and the top side 14 of the assay plate 10, will be the same amount of distance by which the secondary container 24 extends beyond the bottom side 16 of the assay plate 10. This way, multiple assay plates 10 may be stacked upon each other, with the secondary containers 24 of each plate being directly adjacent to and in fluid communication with each other (as described below). This relationship is illustrated in FIG. 7.

Referring now to FIG. 4, in certain other embodiments of the invention, the bottom side 16 of the assay plate 10 comprises a recessed portion 32 around the secondary container(s) 24, such that the secondary container(s) 24 extends beyond the bottom side 16 of such assay plate 10 (at the point where such bottom side surrounds the secondary container(s) 24). In each of the embodiments described above, the secondary container(s) 24 included in the well(s) 18 will be open at both ends, i.e., at the opening within the well 18 (at the top end of each secondary container 24) and at an opening located at (or below) the bottom side 16 of the assay plate 10 (at the bottom end of each secondary container 24).

As described further below, according to other embodiments of the invention, the secondary containers 24 will span the exact thickness of the assay plate 10, e.g., from the top side 14 to the bottom side 16 of the assay plate 10. This embodiment may also be useful when the assay plates 10 are stacked upon each other, such that the secondary containers 24 (of separate assay plates 10) are placed directly adjacent to each other, thereby allowing the secondary containers 24 of multiple assay plates 10 to be simultaneously filled with a reagent (as described below).

Referring to FIG. 4, the side surface 20 of the well preferably comprises a notch 34 (or an alignment feature), which is configured to receive and be positioned adjacent to a correspondingly configured aligning element 36 of the dispenser plate 12, such that when the notch 34 of the well 18 and the aligning element 36 of the dispenser 12 are fittingly positioned next to each other, the dispenser 12 is properly aligned and positioned adjacent to the top side 14 of the assay plate 10. According to certain embodiments of the present invention, the notch 34 located in the side surface 20 of the wells 18 exhibits an angle ranging from 225 to 270 degrees, relative to a plane that runs tangential to the top side 14 of the assay plate 10. In addition to the notch 34 (alignment feature) described above, the invention encompasses other (potentially alternative) mechanical means for ensuring that the dispenser 12 is properly aligned and positioned adjacent to the top side 14 of the assay plate 10. For example, the sides of the dispenser 12 may include tabs that are configured to be received by an aperture (or set of apertures) in the assay plate 10, such that when the tabs are inserted into such apertures, the dispenser 12 is properly aligned and positioned adjacent to the top side 14 of the assay plate 10.

Still referring to FIG. 4, the invention provides that the dispenser plate 12 comprises at least one reservoir 38 (and preferably multiple reservoirs 38), which is in fluid communication with the portion of the secondary container 24 that protrudes through the bottom surface 22 of the well and into an interior volume thereof (i.e., the top end of the secondary container 24), when the dispenser 12 is positioned adjacent to the top side 14 of the assay plate 10, as illustrated in FIGS. 3 and 4. The invention provides that the same reagent may be provided to each reservoir 38 of the dispenser 12 or, alternatively, different reagents may be provided to such reservoirs 38. This way, if desirable, the same well 18 may be provided with multiple types of reagents, with different reagents being provided to the different secondary containers 24 of a particular well 18 via the separate reservoirs 38 of the dispenser 12.

The invention provides that the reservoirs 38 of a dispenser 12 may be filled with a reagent, such that the dispenser 12 may then be aligned with and placed over an assay plate 10 (or set of stacked assay plates 10), in order to then fill the secondary containers 24 of such assay plate(s) 10 as described herein. Alternatively, the dispenser 12 may first be aligned with and placed over an assay plate 10 (or set of stacked assay plates 10), and then filled with a reagent, which will then travel from the dispenser 12 and into the secondary containers 24 of the assay plate(s) 10.

According to certain preferred embodiments of the present invention, the assay plates 10 of the present invention will comprise a plurality of wells 18—each of which may have one or more secondary containers 24 and, preferably, will comprise multiple secondary containers 24. The invention provides that such plates 10 may exhibit a standard number of wells 18, such as 96, 384, and 1536 wells. The volume of liquid that such wells may hold will vary depending on the internal geometries thereof; however, non-limiting examples of such volumes include about 360 microliters per well (for a 96-well plate), 120 microliters per well (for a 384-well plate), and 13 microliters per well (for a 1536-well plate)—less the amount of volume that the protruding portions of the secondary containers 24 will occupy. In addition, the plurality of wells 18 of an assay plate 10 may be organized in various ways and exhibit a variety of configurations, such as 1×8 arrays, 1×12 arrays, 8×12 arrays, 16×24 arrays, and a 32×48 arrays. Still further, as described herein, each of such wells 18 may comprises a plurality of secondary containers 24, such as 4, 8, 10 or 30 secondary containers in each well 18.

The invention provides that the beginning part of a multiplex assay may be carried out and set up using the assemblies described herein, by placing the dispenser 12 on the top side 14 of an assay plate 10, as illustrated in FIG. 3. As described herein, the dispenser 12 will comprise one or more reservoirs 38 (FIG. 4), which is configured to be positioned on the top side 14 of the assay plate 10, such that the reservoirs 38 are in fluid communication with the secondary containers 24. This way, a desired reagent or other liquid may be loaded (filled) into a certain secondary container 24, or group of secondary containers 24, by dispensing an appropriate volume of such reagent or other liquid into the reservoir(s) 38 above the target secondary container(s) 24.

The invention provides that a liquid core waveguide may be formed within the secondary containers 24 when filled with a reagent, when the refractive index of such reagent (which is often aqueous) is higher than the refractive index of the material that forms the secondary container 24. Although a liquid core waveguide may not be formed in many embodiments of the present invention, it is possible to achieve a liquid core waveguide when a material that exhibits a low refractive index is used to construct the secondary container 24, such as Teflon AF, and/or by dispensing a reagent having a high refractive index into the secondary container 24, such as a glycerol-based reagent.

As illustrated in FIG. 4 and mentioned above, the invention contemplates that different types of reagents or other liquids may be loaded into separate reservoirs 38 of the dispenser 12 and, therefore, into separate secondary containers 24 of an assay plate 10. Of course, the type of reagent(s) added to the secondary containers 24 (and primary well 18) of an assay plate 10 will depend on the type of assay being performed. The invention does contemplate that, in addition to traditional reagents, such reagents may include micro-beads (including, without limitation, magnetic micro-beads). The invention provides that multiplexing may be achieved by, for example, (1) dispensing receptors (capture agents) into the individual secondary containers 24; (2) allowing such receptors (capture agents) to become bound to or immobilized on the interior surface of the secondary containers 24 (or otherwise immobilized within the secondary containers 24 via magnetic or other forces, e.g., immobilizing the receptors (capture agents) to certain beads that are retained within the secondary containers 24); (3) decanting such reagents out of the assay plate 10 (while the immobilized receptors (capture agents) remain bound to the interior sides of the secondary containers 24 or otherwise retained therein via other forces); and (4) then dispensing the test samples into the primary wells 18, whereupon the test samples will enter the secondary containers 24 via capillary action and be allowed to interact with the immobilized receptors (capture agents). According to such example, after the test samples are decanted from the assay plate 10, a specific secondary binding agent (i.e., the detection agent) may then be added to the assay plate 10 (secondary containers 24) in order to detect (and potentially quantify) agents, e.g., proteins, that were present in the sample and which bound to the immobilized receptors (capture agents). The detection agent may be tethered to a molecule or agent, e.g., a fluorescent tag, which may be detected using standard instrumentation.

The invention provides that desired reagents or other liquids will travel from the reservoirs 38 of the dispenser 12 and into the secondary containers 24 of an assay plate 10 by way of capillary forces. The invention provides that the protruding edges 48 (FIG. 4) at the top ends of the secondary containers 24 may be inserted into the separate reservoirs 38 of the dispenser 12. For example, FIG. 4 shows a cross-sectional view of one well 18, with two secondary containers 24 and a dispenser 12, with the two reservoirs 38 being aligned and in place for dispensing two separate reagents into the two secondary containers 24. Upon inserting the protruding edges 48 of a secondary container 24 into a separate reservoir 38 of the dispenser 12 that is filled with a reagent, the reagent will travel from the reservoir 38 (and be pulled) into the secondary container 24 by capillary action. Alternatively, the invention provides that placing the secondary containers 24 directly adjacent to the reservoirs 38 of the dispenser 12 will also cause reagent to travel from the dispenser 12 and into the secondary containers 24 by capillary action. The reagent will stop flowing when the dispenser 12 is removed from the assay plate 10, or when the secondary container 24 becomes full, due to the capillary barrier that will form at the bottom ends of the secondary containers 24, e.g., the protruding opening located at the bottom side of a recessed portion 32 of the assay plate 10.

The invention provides that the volume of reagent (or other liquid) that the secondary containers 24 of the assay plate 10 will hold may be determined based on the internal volume of the secondary containers 24. For example, in the case of cylindrically-configured secondary containers 24, the volume of such containers may be calculated using the following formula:

Volume=π×r²×h (pi×radius-squared×height)

Although the secondary containers 24 are illustrated to be cylindrical (and to therefore have a circular cross-section) in FIGS. 1-7, the invention provides that the secondary containers 24 may exhibit other geometries.

The invention provides that when the secondary containers 24 are cylindrical, the secondary containers 24 will exhibit a diameter of about 1 millimeter (or less) or, alternatively, may exhibit a diameter of 500 micrometers, 200 micrometers, or 100 micrometers (or other diameters within such ranges). When the secondary containers 24 are configured in such manner, surface tension forces dominate liquid behavior, and will cause reagents loaded into the secondary containers 24 to be pulled into and contained within the secondary containers 24. A capillary barrier will retain the reagent within the secondary containers 24, until otherwise drawn therefrom by force (e.g., during a reagent decanting step) or by making contact with another secondary container 24 of another assay plate 10, e.g., when multiple assay plates 10 are stacked upon each other (as described below). The invention provides that the reagent will not leak from the secondary containers 24 as a result of these capillary forces (capillary barriers), as illustrated in FIGS. 6 and 7, even if (during the dispensing of reagent into the secondary containers 24) the reservoirs 38 of the dispenser 12 contain a volume of reagent that exceeds the internal volume of the secondary containers 24.

According to certain embodiments of the invention, the top protruding end 28 of the secondary containers 24 will comprise at least one notch, which is configured to facilitate dispensed sample traveling from the well 18 and into the secondary containers 24. The notch will preferably run from a top end 28 of the secondary containers 24 to the bottom of the well 18 or, alternatively, the notch may run from a top end 28 of the secondary containers 24 and terminate at a point before the bottom of the well 18. The invention provides that the notch may be V-shaped, U-shaped, or of any other suitable geometry. According to such embodiments, the bottom side of the reservoirs 38 in the dispenser 12 will preferably comprise a protruding element that corresponds to the notch of the secondary containers 24. For example, if the secondary containers 24 include a V-shaped or U-shaped notch at the top end 28 of the protruding portions thereof, the bottom side of the reservoirs 38 in the dispenser 12 will comprise V-shaped or U-shaped protruding elements, respectively, which may be fittingly inserted into such notches of the secondary containers 24. The engaging relationship between such notches of the secondary containers 24 and the protruding elements of the reservoirs 38 in the dispenser 12, will serve to ensure that the dispenser 12 is properly aligned with and placed over an assay plate 10 when reagents are dispensed. According to similar embodiments, when multiple assay plates 10 are stacked upon each other (as described below), the bottom ends of the secondary containers 24 may comprise a protruding element as described above, which is configured to mate with a notch in the top end 28 of a secondary container 24 of the assay plate 10 upon which it is stacked.

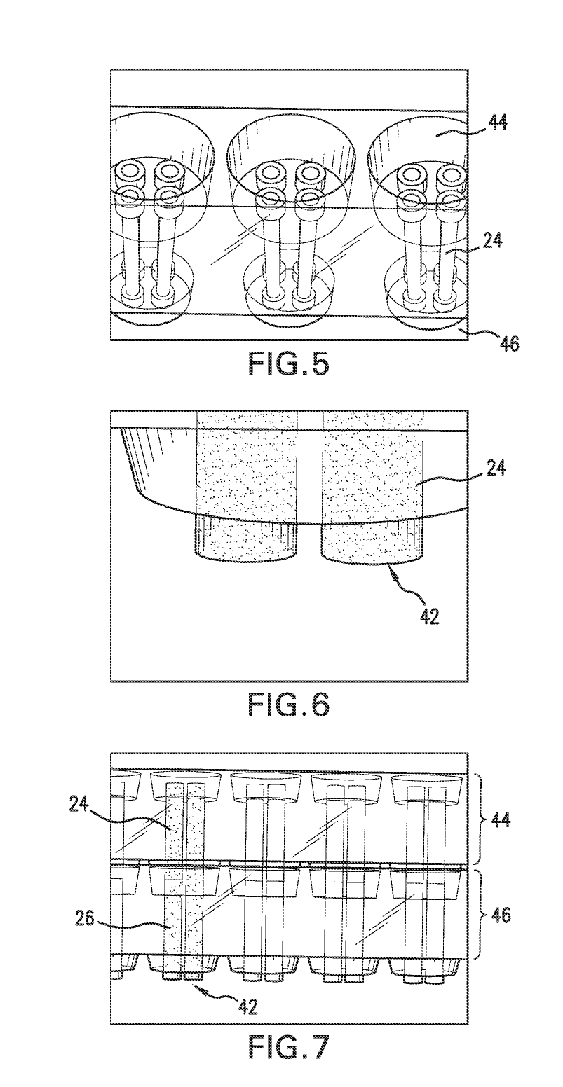

According to certain embodiments of the invention, and referring to FIGS. 5 and 7, a first assay plate 44 (or bar of wells 18) may be stacked on top of a second assay plate 46 (or a second bar of wells 18). According to such embodiments, the plates 44,46 are preferably stacked upon each other such that the secondary containers 24 of each of the first and second plates 44,46 are sufficiently close to each other, such that the capillary barriers of both secondary containers 24 are broken (at the interface between the plates 44,46)—and both plates are consequently in fluid communication with each other. The invention provides that when the secondary containers 24 of the first and second plates 44,46 make physical and aligned contact with each other, fluid will travel from the secondary container 24 of the first assay plate 44 to the secondary container 24 of the second assay plate 46, such that there is no leakage of fluid at the interface between the sets of secondary containers 24 of the first and second plates 44,46. According to such embodiments, the secondary containers 24 of multiple (and identically configured) assay plates 10 may be simultaneously loaded with the desired reagent. This may allow a researcher to quickly fill (load) the secondary containers 24 of multiple (and identically configured) assay plates 10, which will translate into considerable labor savings. In addition, such methodology will ensure that reagent concentrations—which are provided to the secondary containers 24 of multiple (and identically configured) assay plates 10—will be identical. This will provide more precise and consistent experimental results across multiple assay plates 10.

As explained above, when assay plates 10 are stacked upon each other, the secondary containers 24 may span the entire thickness of the assay plates 10, i.e., from the top side 14 to the bottom side 16 of such assay plates 10. In other embodiments, as illustrated in FIG. 7, the top end 28 of the secondary containers 24 may protrude into the interior of the well 18, but end prior to reaching a plane that runs tangential to the top side 14 of the assay plate 10. In such embodiment, the distance between the top end 28 of each secondary container 24, and the top side 14 of the assay plate 10, will be the same amount of distance by which the secondary container 24 extends beyond the bottom side 16 of the assay plate 10. This way, multiple assay plates 44,46 may be stacked upon each other, with the secondary containers 24 of each plate being directly adjacent to and in fluid communication with each other (as illustrated in FIG. 7).

The invention provides that when multiple assay plates 10 are stacked in the foregoing manner, the assay plates 10 may exhibit any configuration of wells 18 (and secondary containers 24 included therein), as long as each of the stacked assay plates 10 exhibit the same configuration. In certain embodiments, for example, the multiple assay plates 10 will exhibit any of the following configurations of wells: 1×8 array, 1×12 array, 1×16 array, 1×24 array, 1×32 array, or 1×48 array. Similar to the other embodiments described herein, each well 18 may comprise a plurality of secondary containers 24, such as 4, 8, 10 or 30 secondary containers 24 in each well 18.

In view of the foregoing, the invention provides that each secondary container 24 may generally consist of a capillary tube that comprises a top end 28 that (1) begins at the top side 14 of the assay plate 10 or (2) begins at a location that protrudes through the bottom surface 22 and into an interior volume of a well 18, but short of the top side 14 of the assay plate 10. Still further, the invention provides that the top end 28 of a secondary container 24 may begin at the bottom surface 22 of the well 18 (such that it does not protrude into the interior of the well 18). Such configurations for the top end 28 of a secondary container 24 may be combined with a variety of configurations for the bottom end thereof, namely, the bottom end of a secondary container 24 may end (1) at the bottom side 16 of the assay plate or (2) at a location that extends beyond the bottom side 16 of the assay plate 10 (as described above). Alternatively, the invention provides that the bottom end of a secondary container 24 may terminate at a location that is between the bottom surface 22 of the well 18 and the bottom side 16 of the assay plate 10.

According to certain additional embodiments of the invention, the secondary containers 24 of the assay plates 10 may be provided with reagent through forces other than capillary action. For example, reagent may be dispensed into the secondary containers 24 through mere gravitational forces or, alternatively, a reagent may be dispensed therein using an external pressure source (from a pressurized dispenser 12 or other source of reagent, such as single or multiple pipettes).

According to yet further embodiments of the present invention, the secondary containers 24 may comprise a restriction located at (or near) the bottom ends of the secondary containers 24. The restriction will preferably be effective to retain liquid inside of the secondary containers 24 through capillary forces. This restriction may be integrally formed with the secondary containers 24. Alternatively, the restrictions may be applied, when needed, to the bottom ends of the secondary containers 24 during the performance of an assay.

According to such embodiments, the “restriction” may comprise, by way of example and not limitation, a narrowing of the bottom end of the secondary container 24 (to reduce the size/diameter of the aperture at the bottom end thereof to encourage a capillary barrier). Alternatively, the restriction may comprise a circular disc, which includes an aperture smaller than the aperture of the secondary container 24, which may be applied to the bottom end of the secondary container 24. Such a disc may be made out of hydrophobic material or be coated with a hydrophobic layer. Still further, the restriction may consist of a grid, with a mesh size smaller than the aperture of the secondary container 24, which may be applied to the bottom end of the secondary container 24. This geometry is advantageous insofar as it only requires a relatively low precision alignment, relative to the end of the secondary container 24. Such a grid may be comprised of hydrophobic material or be coated with a hydrophobic layer. In addition, the restriction may comprise a porous membrane that may be applied to the bottom end of the secondary container 24. According to yet further non-limiting examples, the restriction may comprise a plate, which includes an array of features that may be applied to the end of at least one secondary container 24. The dimensions of this array will preferably match the dimensions of the plate, with the secondary containers 24 positioned such that both plates may be aligned respective to each other—with the flow restrictive features being applied to the end of all secondary containers 24. These features may protrude from the surface of the plate, so that they may be easily positioned in a manner that is adjacent to the end of the secondary containers 24.

Both the dispenser 12 (comprising the reservoirs 38) and the assay plates 10 described herein may be fabricated at low cost using plastic injection molding. The dispenser 12 and assay plates 10 may be comprised of polystyrene, polypropylene, polycarbonate, or other suitable materials. Still further, the invention provides that the assay plates 10 may consist of multiple materials. For example, a majority of a plate may be manufactured from one of the plastics listed above, whereas the secondary containers 24 (or just the internal surface areas thereof) may be comprised of metals, glass, or other materials, e.g., by inserting a separate sleeve or tubing into such secondary containers 24. The molds that are necessary to fabricate such plates could be made by high-resolution machining, laser machining or micro-fabrication techniques to achieve the required precision.

In addition to the assay plate assemblies described herein, the present invention further encompasses the assay plates described herein, without the dispensers described above. In addition, methods of using the assay plates (and assay plate assemblies) for carrying out multiplex binding assays are encompassed by the present invention. The multiplex assay assemblies (and methods of use thereof) allow for the multiplexing of small volume samples—and for the separate dispensing of the reagents required by each of the multiplex assays. This way, each assay can be optimized individually, which leads to better assay quality (both in terms of reproducibility and sensitivity), and renders the modification of an assay panel possible without requiring the re-optimization of the entire panel.

Although certain example methods, apparatus, and/or articles of manufacture have been described herein, the scope of coverage of this disclosure is not limited thereto. On the contrary, this disclosure covers all methods, apparatus, and/or articles of manufacture fairly falling within the scope of the appended claims—either literally or under the doctrine of equivalents.

What is claimed is:

1. An assay plate assembly, which comprises:
(a) an assay plate that comprises a top side, a bottom side, and at least one well accessible from the top side of the plate, wherein the well comprises a side surface and a bottom surface, wherein at least one secondary container protrudes through the bottom surface and into an interior volume of the well; and
(b) a dispenser plate that is adapted to be positioned adjacent to the top side of the assay plate and to provide one or more reagents to one or more secondary containers of at least one well of the assay plate.

2. The assay plate assembly of claim 1, wherein the secondary container comprises a capillary tube that (a) begins at a location that protrudes through the bottom surface and into an interior volume of the well and (b) ends at a location that extends beyond a bottom side of the assay plate.

3. The assay plate assembly of claim 2, wherein the bottom side of the assay plate comprises a recess around each secondary container at the location that extends beyond the bottom side of such assay plate.

4. The assay plate assembly of claim 3, wherein the side surface of the at least one well comprises a notch, which is configured to receive and be positioned adjacent to a correspondingly configured aligning element of the dispenser plate, such that when the notch of the well and the aligning element of the dispenser are fittingly positioned next to each other, the dispenser is properly aligned and positioned adjacent to the top side of the assay plate.

5. The assay plate assembly of claim 4, wherein the notch located in the side surface of the at least one well exhibits an angle ranging from 225 to 270 degrees, relative to a plane that runs tangential to the top side of the assay plate.

6. The assay plate assembly of claim 3, wherein the dispenser plate comprises at least one reservoir, which is in fluid communication with a portion of the at least one secondary container that protrudes through the bottom surface and into an interior volume of the well, when the dispenser is positioned adjacent to the top side of the assay plate.

7. An assay plate assembly, which comprises:
(a) a first assay plate that comprises a top side, a bottom side, and a plurality of wells accessible from the top side of the plate, wherein each well of the plurality of wells comprises a side surface and a bottom surface, wherein at least one secondary container is included within each well; and
(b) a dispenser plate that is adapted to be positioned adjacent to the top side of the assay plate and to provide one or more reagents to one or more secondary containers of at least one well of the assay plate.

8. The assay plate assembly of claim 7, wherein each secondary container comprises a capillary tube that spans a thickness of the assay plate, which begins at the top side of the assay plate and ends at the bottom side of the assay plate.

9. The assay plate assembly of claim 8, wherein a top end of each secondary container comprises at least one notch, which is configured to receive a correspondingly configured protruding element located at a bottom end of a reservoir that is included in the dispenser plate.

10. The assay plate assembly of claim 8, wherein the side surface of each well comprises a notch, which is configured to receive and be positioned adjacent to a correspondingly configured aligning element of the dispenser plate, such that when the notch of a well and the aligning element of the dispenser are fittingly positioned next to each other, the dispenser is properly aligned and positioned adjacent to the top side of the assay plate.

11. The assay plate assembly of claim 10, wherein the notch located in the side surface of each well exhibits an angle ranging from 225 to 270 degrees, relative to a plane that runs tangential to the top side of the assay plate.

12. The assay plate assembly of claim 7, wherein the dispenser plate comprises at least one reservoir, which is in fluid communication with a portion of the at least one secondary container when the dispenser is positioned adjacent to the top side of the assay plate.

13. The assay plate assembly of claim 12, wherein the plurality of wells of the assay plate comprises a configuration selected from the group consisting of a:
(a) 1×8 array;
(b) 1×12 array;
(c) 8×12 array;
(d) 16×24 array; and
(e) 32×48 array.

14. The assay plate assembly of claim 12, wherein each of the plurality of wells of the assay plate comprises:
(a) 4 secondary containers;
(b) 8 secondary containers;
(c) 10 secondary containers; or
(d) 30 secondary containers.

15. The assay plate assembly of claim 7, which further comprises a second assay plate that exhibits a same configuration as the first assay plate, wherein (a) the second assay plate is stacked directly on top of the first assay plate, such that the top side of the first assay plate makes contact with and is directly adjacent to a bottom side of the second assay plate; and (b) the secondary containers of the first assay plate are in fluid communication with the secondary containers of the second assay plate.

16. The assay plate assembly of claim 7, wherein the at least one secondary container comprises a restriction at its bottom end, which is effective to retain and hold the one or more reagents dispensed therein, wherein the restriction may be (a) integrally formed with the secondary container or (b) applied to the bottom end of the secondary container during use of the assay plate.

17. The assay plate assembly of claim 7, wherein the at least one secondary container is adapted to receive the one or more reagents when the reagents are dispensed therein via gravity or through an external pressure source.

18. An assay plate, which comprises a top side, a bottom side, and a plurality of wells accessible from the top side of the plate, wherein each well of the plurality of wells comprises a side surface, a bottom surface, and at least one secondary container.

19. The assay plate of claim 18, wherein each secondary container consists of a capillary tube that comprises:
(a) a top end that exhibits one of the following configurations:
(i) the top end of the secondary container begins at the top side of the assay plate; or
(ii) the top end of the secondary container begins at a location that protrudes through the bottom surface and into an interior volume of a well, but short of the top side of the assay plate; or
(iii) the top end of the secondary container begins at the bottom surface of the well; and
(b) a bottom end that exhibits one of the following configurations:
(i) the bottom end of the secondary container ends at the bottom side of the assay plate; or
(ii) the bottom end of the secondary container ends at a location that extends beyond the bottom side of the assay plate; or
(iii) the bottom end of the secondary container ends at a location that is between the bottom surface of the well and the bottom side of the assay plate.

20. The assay plate of claim 19, wherein the bottom side of the assay plate comprises a recess around each secondary container, in each well, at the location of the secondary container that extends beyond the bottom side of such assay plate.
